(19) United States
(12) Patent Application Publication
(10) Pub. No.: US 2014/0236335 A1
(43) Pub. Date: Aug. 21, 2014

(54) 3D Scanning, Modeling and Printing for the Creation of Custom-Fitted Ostomy Devices

(71) Applicants: Frank Allen Lewis, St Petersburg, FL (US); John Arthur Accardi, Tampa, FL (US)

(72) Inventors: Frank Allen Lewis, St Petersburg, FL (US); John Arthur Accardi, Tampa, FL (US)

(21) Appl. No.: 14/182,135

(22) Filed: Feb. 17, 2014

Related U.S. Application Data
(60) Provisional application No. 61/765,117, filed on Feb. 15, 2013.

(51) Int. Cl. B29C 67/00; G06F 17/50; A61F 5/445

(52) U.S. Cl. CPC B29C 67/0051; A61F 5/445; G06F 17/50
USPC 700/98

(57) ABSTRACT

In accordance with this embodiment of the application of a 3D scanning, modeling and printing process to create custom-fitted ostomy devices that minimize leakage and odor and maximize mobility and comfort.

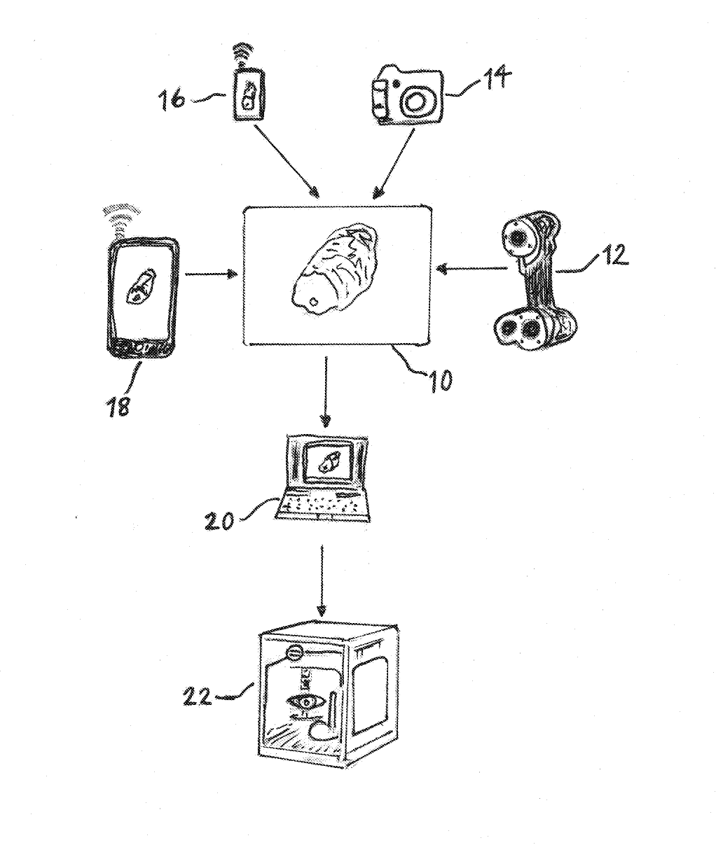

This application claims the benefits of provisional patent applications Ser. No. 61/765,117 filed 2013 Feb 15 by the present inventors.

### BACKGROUND

An ostomy is a surgically-created intestinal (colostomy or ileostomy) or urinary (urostomy) tract diversion that modifies the normal pathway for waste elimination. A colostomy is an abdominal opening from any part of the large intestine (colon), made because part of the colon has been removed or bypassed. An ileostomy is an abdominal opening from the terminal small intestine (ileum), made because the entire colon has been removed or bypassed. A urostomy is an abdominal opening from the urinary tract.

For all ostomy surgeries, an opening called a stoma is made through the abdominal wall. An ostomy device (wafer and attached pouch) is situated over the stoma to collect waste that flows through and out of the stoma. It is worn at all times and must be periodically emptied and replaced. A person who wears an ostomy device is called an ostomate. This therapeutic approach can be temporary or permanent and creates many quality of life challenges.

In many cases, wearing current ostomy devices leads to intensified physical and emotional distress, discomfort, and a diminished quality of life. Ostomates report a high percentage of skin irritation (76%), pouch leakage (62%), offensive odor (59%), depression/anxiety (53%) and a reduction in pleasurable activities (54%) using currently available ostomy devices.

While many types of ostomy devices are available to ostomates, state-of-the-art design technology and materials applications are sorely lacking Current ostomy devices include pouches, wafers, barriers, belts, deodorizers, and seals that are bulky, uncomfortable and prone to leakage of malodorous solids, liquids and gases. The ostomy devices are worn 24 hours a day/seven days a week, making it difficult to travel, empty or change the device. The ostomy industry offers these devices in standard stoma diameters or places the responsibility on an ostomate to cut an opening in an ostomy device to get the best fit around the stoma.

Current ostomy devices are manufactured using a traditional subtractive process of cutting away material to make molds and prototypes to create a final product. This traditional approach can be costly and time consuming and thus limits production to standardized designs and standardized sizes.

### SUMMARY

In accordance with this embodiment a process to create custom-fitted ostomy devices comprises the application of 3D scanning, modeling and printing technologies.

### ADVANTAGES

Unlike traditional manufacturing processes, recent advances in 3D scanning, modeling and printing technologies are transforming production capabilities by enhancing product design, offering direct digital manufacturing, and facilitating personal fabrication and quick turn-around times. The 3D scanning, modeling and printing process applies these modern fabrication and materials technologies to create personalized custom-fitted ostomy devices that address important quality of life issues. The custom-fitted ostomy device minimizes leakage and odor and maximizes mobility and comfort. The process can always be re-visited to accommodate future changes in an ostomate's stoma size and shape.

### DRAWING

Drawing shows a flowchart of the 3D scanning, modeling and printing process for the creation of custom-fitted ostomy devices.

### DRAWINGS—REFERENCE NUMERALS

10—scanned stoma image
12—handheld scanner
14—digital camera
16—smart phone
18—smart pad
20—3D modeling computer & software
22—3D printer

### DETAILED DESCRIPTION

The 3D scanning, modeling and printing process is a computerized state-of-the-art technology and materials application for the design, manufacture and production of custom-fitted ostomy devices. The scanning process includes but is not limited to 3D portable, handheld, and fixed unit scanners 12, digital cameras 14, smart phones 16, smart pads 18 and other imaging technologies to record multiple perspective images of an ostomate's stoma and surrounding tissue to create a 3D model design of the scanned area.

The composite image or 3D model design 10 is uploaded to a computer with 3D modeling and design software 20. The resulting 3D model design of the stoma and surrounding tissue is edited and integrated into the ostomy device design. This integration process includes but is not limited to creating a stoma sleeve where the sleeve's interior is an exact copy of the exterior structure and surface of the stoma and surrounding tissue. This results in a stoma sleeve that is perfectly fitted for an ostomate's stoma and surrounding tissue. The personalized custom-fitted design creates a more stable, comfortable, leak-resistant and odor-resistant ostomy device.

The final integrated 3D model design is uploaded to a 3D printer 22 to manufacture a custom-fitted ostomy device. The 3D printing process includes but is not limited to selective laser sintering and fusion deposition 3D printing technologies. Selective laser sintering and fused deposition are additive processes that use the melting of fine powders or softening of a polymer to produce a 3D shape by adding material in layers. There is an array of materials available to produce the ostomy device including but not limited to various plastics, polymer resins, metals, and glasses. The process can be re-visited to accommodate future changes in stoma size and shape.

### OPERATION—DRAWING OF FLOWCHART

Images of the stoma and surrounding tissue are captured using a handheld scanner 12, or digital camera 14, or smart phone 16, or smart pad 18. The composite image or 3D model design 10 is transmitted wirelessly or by landline to a website, cloud, computer or network 20 containing 3D modeling and design software.

The resulting 3D model design of the stoma and surrounding tissue is edited and integrated into an ostomy device using 3D design software. The custom-fitted ostomy device 3D model design is transmitted wirelessly or by landline to a website, cloud, computer or network linked for production on a 3D printer 22. There is an array of materials available to produce the ostomy device including but not limited to various plastics, polymers, resins, metals, and glasses.

### CONCLUSIONS, RAMIFICATIONS AND SCOPE

Accordingly, the reader will see that the 3D scanning, modeling and printing process distinguishes itself through the application of modern fabrication and materials technologies with the following advantages:

it creates a personalized custom-fitted ostomy device based on the unique shape of an ostomate's stoma and surrounding tissue;

it addresses important quality of life issues;

it minimizes leakage and odor and maximizes mobility and comfort;

it can be re-visited to allow for future changes in stoma size and shape;

it results in the enhancement of public and personal activities and an increased quality of life for ostomates.

I claim:

1. A process for creating custom-fitted ostomy devices, comprising:
a. providing portable, handheld and fixed unit scanners, digital cameras, smart phones, smart pads and other imaging technologies which will:
1. scan and record multiple perspective images of an ostomate's stoma and surrounding tissue to create a composite 3D image, and
2. transmit the composite 3D image wirelessly or by landline to a 3D modeling and design computer,
b. providing a computer with 3D modeling and design software which will:
1. receive the composite images of the stoma and surrounding tissue, and
2. enable editing of the 3D model of the stoma and surrounding tissue, and
3. integrate the 3D model of the stoma and surrounding tissue composite image into an ostomy device design to create a custom-fitted model, and
4. transmit the finished model design wirelessly or by landline to a 3D printer,
c. providing a 3D printer which will:
1. receive the finished custom-fitted 3D ostomy device image and
2. produce a custom-fitted ostomy device made from but not limited to various plastics, polymers, resins, metals, and glasses,
whereby said ostomy devices will be created to provide a custom-fit.
